(19) United States
(12) Patent Application Publication
(10) Pub. No.: US 2018/0050147 A1
(43) Pub. Date: Feb. 22, 2018

(54) HEMODIALYSIS DEVICE

(71) Applicant: Hsien-Chang SHIH, Taipei (TW)

(72) Inventor: Hsien-Chang SHIH, Taipei (TW)

(21) Appl. No.: 15/448,016

(22) Filed: Mar. 2, 2017

(30) Foreign Application Priority Data
Aug. 19, 2016 (TW) 105126529

(51) Int. Cl. A61M 1/36; A61M 25/00

(52) U.S. Cl. CPC A61M 1/3659; A61M 25/003; A61M 25/007; A61M 2205/3317; A61M 2205/3523; A61M 2205/3368; A61M 2205/50; A61M 2025/0031

(57) ABSTRACT

A hemodialysis device which includes a main tube, a first linking tube, a second linking tube and a third linking tube is disclosed. The main tube includes a central tube, an annular tube, a plurality of dialysis holes, a first end and a second end. The central tube includes a blood passage. The annular tube includes a waste passage which surrounds the central tube. The plurality of dialysis holes are located on the central tube, wherein the blood passage is connected to the waste passage via the plurality of dialysis holes. The first end and the second end are the two opposite ends of the main tube. The first linking tube is connected to the first end and to the central tube. The second linking tube is connected to the second end and to the central tube.

### BACKGROUND OF THE INVENTION

### 1. Field of the Invention

The present invention relates to a hemodialysis device; more particularly, the present invention relates to a hemodialysis device which is convenient to use and can be used to perform dialysis on a patient with kidney failure at any time.

### 2. Description of the Related Art

Uremia is an illness of a patient with kidney failure in which urea and waste cannot be excreted, such that the urea and the waste will remain in the human body and cause poisoning. If the uremia is serious, the metabolism of the patient will be disrupted such that the organs of the patient cannot work normally, and the patient may go into shock or die. Therefore, if the patient suffers kidney failure, the patient must choose one of three traditional therapies for preventing uremia: kidney transplant, peritoneal dialysis, or blood dialysis. In a kidney transplant, a functioning kidney is obtained from an organ donor who has two functioning kidneys, and this functioning kidney is transplanted into the body of the patient to replace the function of the failed kidney. In peritoneal dialysis, a permanent catheter is installed in the abdomen of the patient, and a dialysis solution is injected via the catheter into the peritoneum so that the peritoneum of the patient can filter the urea and the waste; when the concentration of the waste in the dialysis solution reaches a certain level, the dialysis solution with the waste is extracted from the peritoneum and fresh and clean dialysis solution is injected. In blood dialysis, a permanent fistula is constructed on the artery of the hand of the patient, and the blood of the patient is drawn via the permanent fistula to the outside of the body, where the blood enters a blood dialysis machine and passes through the permeable membrane of the blood dialysis machine to filter out the urea and the waste before being transferred back to the body of the patient via the permanent fistula.

However, the abovementioned three traditional therapies have different problems. The problem with a kidney transplant is that functioning kidneys for transplant are extremely rare. According to the historical statistics of the Taiwan Organ Registry and Sharing Center, the number of kidney transplants every year is between 70 and 130; however, patients with kidney failure in Taiwan number more than 70,000, so the chance of obtaining a suitable kidney for the patient is very poor. In addition, after the kidney transplant operation, the patient must take anti-rejection medicines for the rest of his or her life to prevent rejection of the new kidney by the body of the patient. The problem with peritoneal dialysis is that the dialysis solution with the waste must be extracted and fresh dialysis solution must be injected 4 to 6 times each day, which is very inconvenient for the patient; in addition, the catheter on the abdomen of the patient must be clean, or a virus may easily enter the body via the catheter or the dialysis solution and cause peritonitis. The problem of blood dialysis is that the patient must travel to the hospital for blood dialysis therapy 2 to 3 times per week, and the blood dialysis therapy usually takes 4-6 hours, which is very time-consuming and inconvenient for the patient. In addition, the patient must be given an injection when receiving blood dialysis therapy, so the patient must endure the discomfort of the injection, and the arm of the patient may have a wound caused by the injection. Furthermore, blood dialysis uses an artery of the patient for transferring the blood between the body and the dialysis machine; in the course of time, the artery of the patient may become blocked such that the blood circulation to the extremities of the body (such as the fingers) may be restricted and lead to conditions requiring amputation.

Because the traditional therapies for uremia respectively have different problems, there is a need to provide a new hemodialysis device to solve the problems of the traditional therapies.

### SUMMARY OF THE INVENTION

It is an object of the present invention to provide a hemodialysis device which is convenient to use and which can be used to perform dialysis on a patient with kidney failure at any time.

To achieve the abovementioned object, the hemodialysis device of the present invention includes a main tube, a first linking tube, a second linking tube and a third linking tube. The main tube includes a central tube, an annular tube, a plurality of dialysis holes, a first end and a second end. The central tube includes a blood passage. The annular tube includes a waste passage which surrounds the central tube. The plurality of dialysis holes are located on the central tube, wherein the blood passage is connected to the waste passage via the plurality of dialysis holes. The first end and the second end are the two opposite ends of the main tube. The first linking tube is connected to the first end and to the central tube. The second linking tube is connected to the second end and to the central tube. The third linking tube is connected to the second end and to the annular tube.

According to one embodiment of the present invention, the hemodialysis device further includes a first electrode and a second electrode. The first electrode is located at the first end, and the second electrode is located at the second end. The first electrode and the second electrode form an electric field.

According to one embodiment of the present invention, the hemodialysis device further includes a controlling module, and the controlling module is electrically connected to the first electrode and the second electrode.

According to one embodiment of the present invention, the hemodialysis device further includes a sensor, and the controlling module is electrically connected to the sensor.

According to one embodiment of the present invention, the hemodialysis device further includes a wireless module, and the controlling module is electrically connected to the wireless module.

According to one embodiment of the present invention, the hemodialysis device further includes a power module, and the controlling module is electrically connected to the power module.

According to one embodiment of the present invention, the first linking tube further includes a connecting unit, and the connecting unit is connected to the main tube.

According to one embodiment of the present invention, the first linking tube further includes a first fastening tube; the first fastening tube is located at one end of the first linking tube, and the end of the first linking tube is opposite to the connecting unit.

According to one embodiment of the present invention, the second linking tube further includes a second fastening tube; the second fastening tube is located at one end of the second linking tube, and the end of the second linking tube is away from the main tube.

According to one embodiment of the present invention, the connecting unit is shaped as a funnel.

### BRIEF DESCRIPTION OF THE DRAWINGS

These and other objects and advantages of the present invention will become apparent from the following description of the accompanying drawings, which discloses several embodiments of the present invention. It is to be understood that the drawings are to be used for purposes of illustration only and not as a definition of the invention.

In the drawings, wherein similar reference numerals denote similar elements throughout the several views:

FIG. 1 illustrates a schematic drawing of the hemodialysis device in the human body in one embodiment of the present invention.

FIG. 2 illustrates a schematic drawing of the hemodialysis device connected to the artery and the kidney in one embodiment of the present invention.

FIG. 3 illustrates a sectional view of the hemodialysis device in one embodiment of the present invention.

FIG. 4 illustrates a sectional view of the main tube in one embodiment of the present invention along the section line ZZ shown in FIG. 3.

FIG. 5 illustrates a schematic drawing of the hemodialysis device which forms the electric field in one embodiment of the present invention.

FIG. 6 illustrates a schematic drawing of the direction of the blood flow in the hemodialysis device in one embodiment of the present invention.

FIG. 7 illustrates a schematic drawing of the direction of waste flow and the direction of outflow in the hemodialysis device in one embodiment of the present invention.

FIG. 8 illustrates a schematic drawing of the hemodialysis device on the outside of the human body in one embodiment of the present invention.

FIG. 9 illustrates a system structure drawing of the hemodialysis device in one embodiment of the present invention.

### DETAILED DESCRIPTION OF THE PREFERRED EMBODIMENT

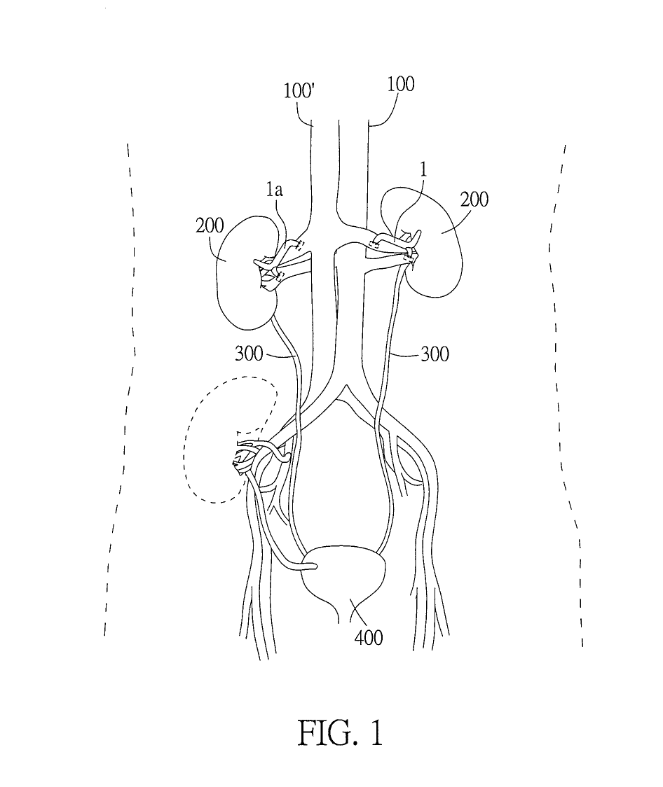

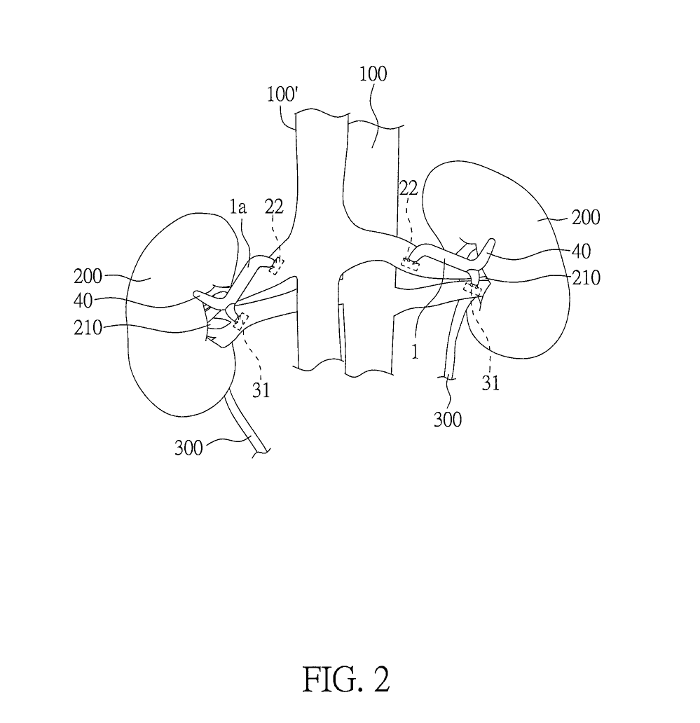

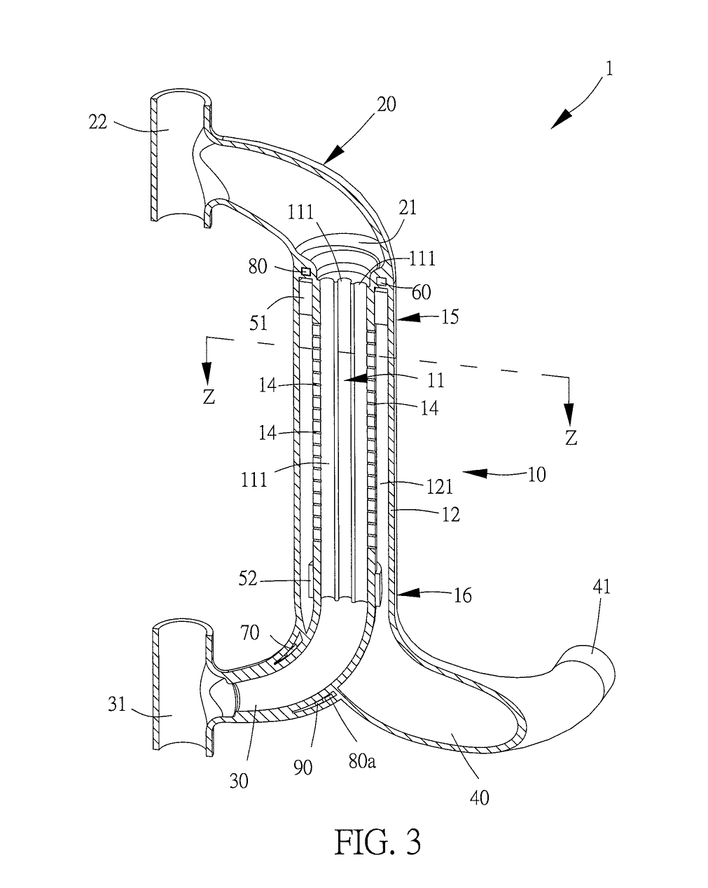

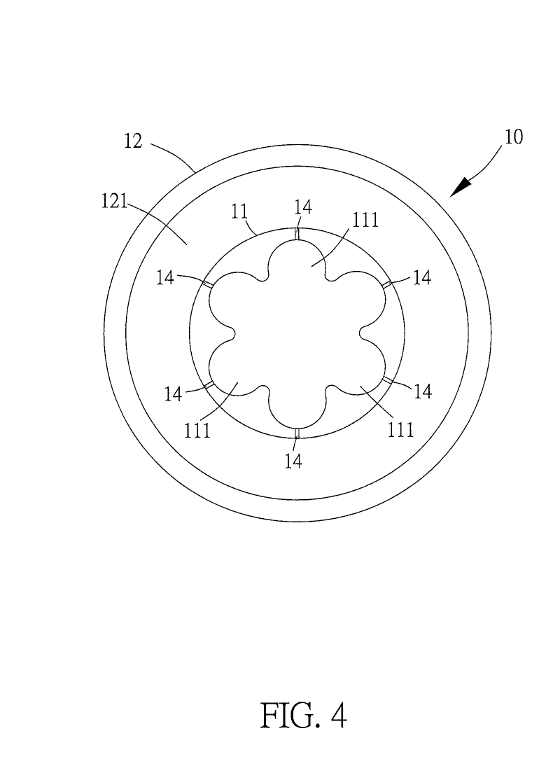

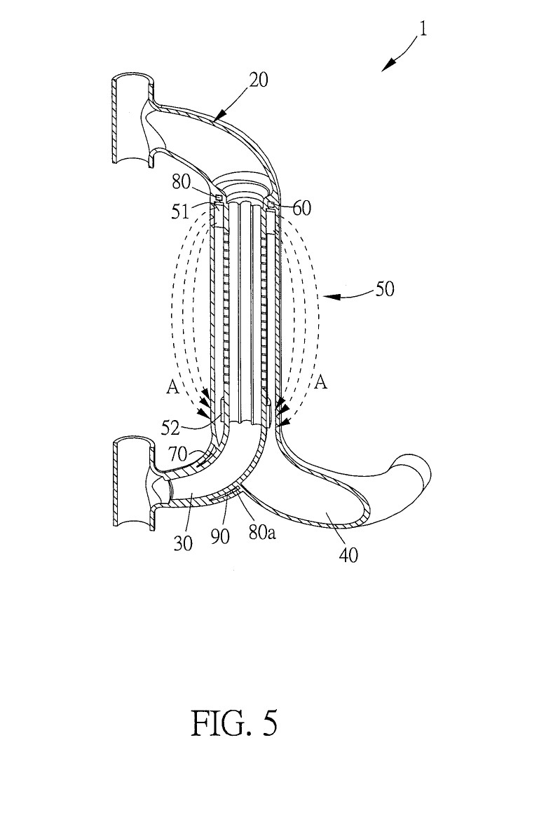

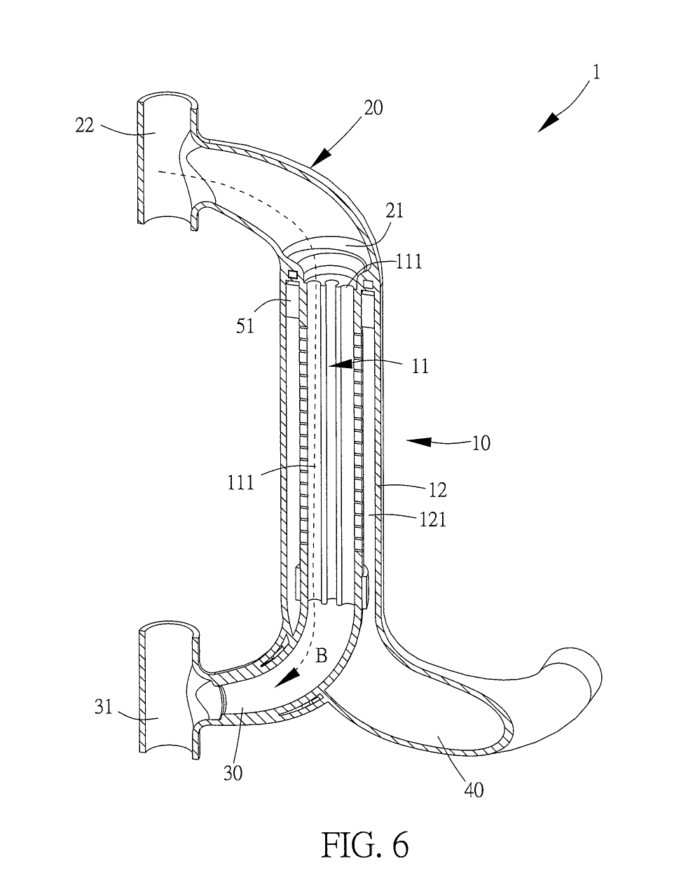

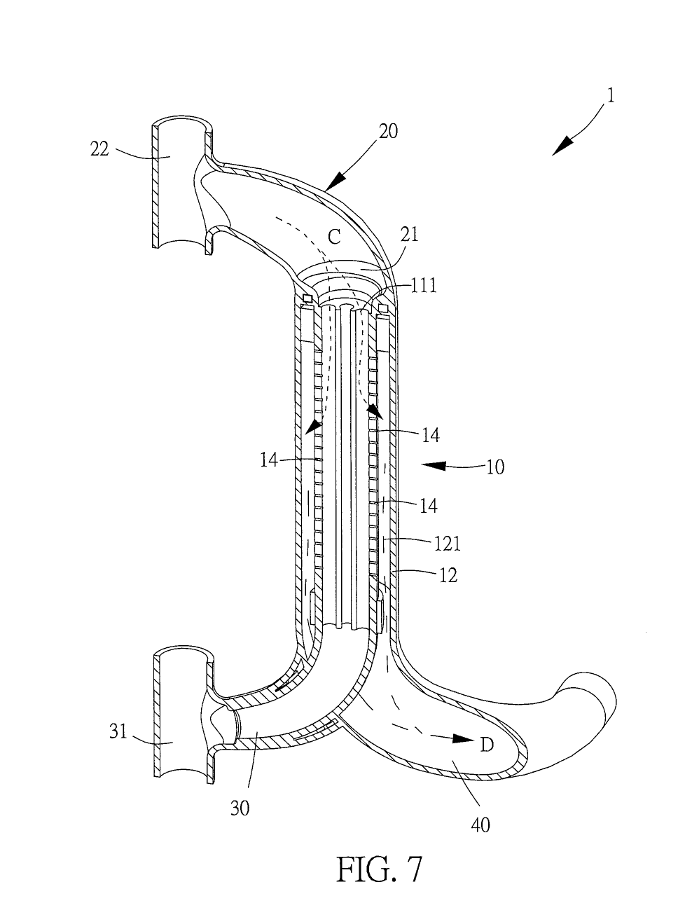

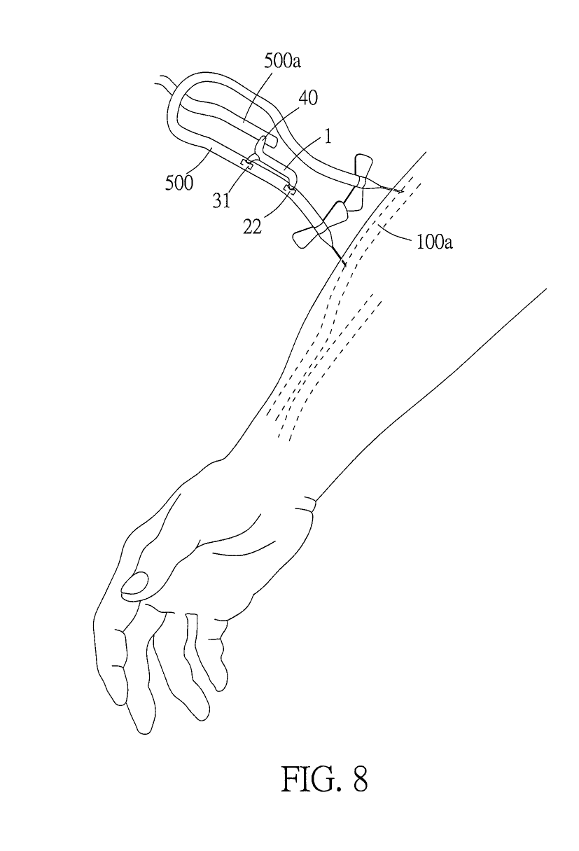

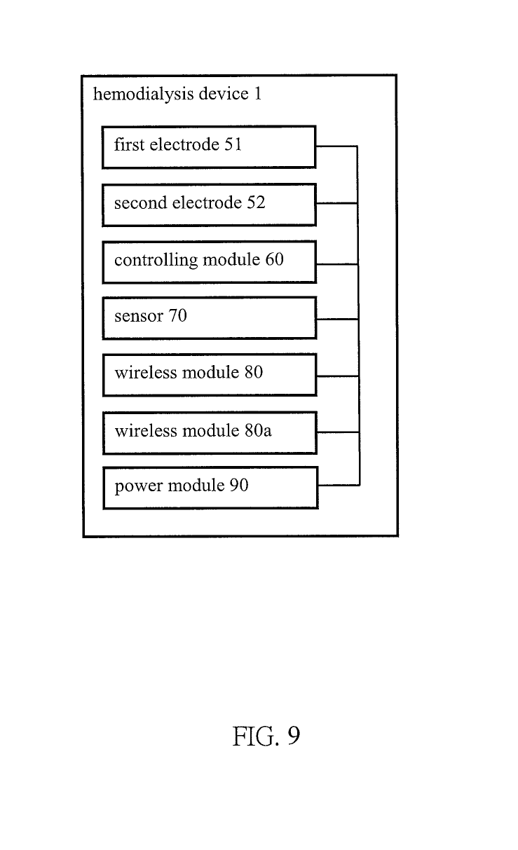

Please refer to FIG. 1 to FIG. 9 regarding the hemodialysis device in one embodiment of the present invention. FIG. 1 illustrates a schematic drawing of the hemodialysis device in the human body in one embodiment of the present invention. FIG. 2 illustrates a schematic drawing of the hemodialysis device connected to the artery and the kidney in one embodiment of the present invention. FIG. 3 illustrates a sectional view of the hemodialysis device in one embodiment of the present invention. FIG. 4 illustrates a sectional view of the main tube in one embodiment of the present invention along the section line ZZ shown in FIG. 3. FIG. 5 illustrates a schematic drawing of the hemodialysis device which forms the electric field in one embodiment of the present invention. FIG. 6 illustrates a schematic drawing of the direction of the blood flow in the hemodialysis device in one embodiment of the present invention. FIG. 7 illustrates a schematic drawing of the direction of waste flow and the direction of outflow in the hemodialysis device in one embodiment of the present invention. FIG. 8 illustrates a schematic drawing of the hemodialysis device on the outside of the human body in one embodiment of the present invention. FIG. 9 illustrates a system structure drawing of the hemodialysis device in one embodiment of the present invention.

As shown in FIG. 1 to FIG. 3, in one embodiment of the present invention, the hemodialysis devices 1, 1a are installed in the human body of a patient with kidney failure to replace the failed kidney 200 and to metabolize the urea and the waste in the human body of the patient. The hemodialysis device 1 of the present invention is made of a polymer material which has high biocompatibility, high toughness and high flexibility. The hemodialysis device 1 includes a main tube 10, a first linking tube 20, a second linking tube 30, a third linking tube 40, a first electrode 51, a second electrode 52, a controlling module 60, a sensor 70, two wireless modules 80, 80a and a power module 90. The hemodialysis device 1a and the hemodialysis device 1 are the same devices and have the same materials, structures and functions; the only difference between the hemodialysis devices 1, 1a is that the hemodialysis devices 1, 1a are two symmetrical structures, so there is no need to furthermore describe the structure and the function of the hemodialysis device 1a.

As shown in FIG. 3 and FIG. 4, in one embodiment of the present invention, the main tube 10 is used for allowing the blood, the body fluids, and the waste and urea in solution in the body fluids of the patient to flow into the main tube 10, and for separating the waste and urea in the body fluids from the clean blood. The main tube 10 includes a central tube 11, an annular tube 12, a plurality of dialysis holes 14, a first end 15 and a second end 16. The central tube 11 is a cylindrical tube for allowing the blood and the body fluids with the waste and the urea of the patient to flow into the central tube 11. The central tube 11 includes a plurality of blood passages 111; the two ends of each of the blood passages 111 are respectively connected to the first linking tube 20 and the second linking tube 30. The blood passage 111 is used for allowing the waste and urea in solution in the body fluids and the blood to flow into the main tube 10 from the first linking tube 20, and for allowing the blood to leave the main tube 10 after the dialysis and flow into the second linking tube 30. However, the amount of the blood passages 111 is not limited to a plural amount; the amount can also be single. The annular tube 12 includes a waste passage 121, and the waste passage 121 surrounds the central tube 11. The waste passage 121 is used for allowing the waste and urea in solution in the body fluids and separated from the blood to flow into the third linking tube 40. The plurality of dialysis holes 14 are located on the central tube 11. The plurality of blood passages 111 are connected to the waste passage 121 via the plurality of dialysis holes 14. The diameter of the dialysis holes 14 is less than the smallest diameter of the leucons (the smallest leucon is the lymphocyte, whose diameter is 9 μm), less than the average diameter of the red blood cells (7 micrometers), and less than the average diameter of thrombocytes (3 micrometers); whereby, the leucons, the red blood cells and the thrombocytes cannot pass through the dialysis holes 14 and cannot enter the waste passage 121 from the blood passages 111; therefore, only the liquid urea and waste in solution in the body fluids can enter the waste passage 121 from the blood passages 111 via the dialysis holes 14. The first end 15 and the second end 16 are two opposite ends of the main tube 10; the first end 15 is the end which is connected to the first linking tube 20, and the second end 16 is the other end, which is connected to the second linking tube 30 and the third linking tube 40.

As shown in FIG. 2, FIG. 3 and FIG. 8, in one embodiment of the present invention, the first linking tube 20 is a tube structure with flexibility; the first linking tube 20 includes a connecting unit 21 and a first fastening tube 22. The connecting unit 21 is shaped as a funnel. The connecting unit 21 is connected to the first end 15 of the main tube 10 and to the central tube 11. Via the funnel structure of the connecting unit 21, the connecting unit 21 can import the blood and the body fluids of the patient into the main tube 10 such that the blood and body fluids in the funnel of the connecting unit 21 will apply pressure to the blood and the body fluids in the main tube 10 such that the blood and the body fluids in the main tube 10 will flow smoothly. The first fastening tube 22 is located on the end of the first linking tube 20, and this end is opposite to the connecting unit 21. The first fastening tube 22 is used for fastening and connecting to the vein 100′ of the patient or for fastening and connecting to the external catheter 500 (which is connected to the artery 100a of the patient), allowing the blood and the body fluids in the vein 100′ or the artery 100a of the patient to flow into the hemodialysis device 1. Because the first linking tube 20 is flexible, the first linking tube 20 can be coordinated with the vein 100′ or the artery 100a of the patient to bend appropriately to fasten to the vein 100′ or the artery 100a, and the first linking tube 20 with toughness can also support the vascular wall to prevent the vein 100′ or the artery 100a from collapsing.

In one embodiment of the present invention, the second linking tube 30 is a tube structure that is flexible. The second linking tube 30 is connected to the second end 16 and connected to the central tube 11. The second linking tube 30 is used for allowing the clean blood to flow into the human body of the patient from the central tube 11. The second linking tube 30 includes a second fastening tube 31. The second fastening tube 31 is located on the end of the second linking tube 30, wherein this end is away from the main tube 10. The second fastening tube 31 is used for fastening and connecting to the artery 100 of the patient, or for fastening and connecting to the external catheter 500 (which is connected to the artery 100a of the patient), such that the blood can flow into the arteries 100, 100a of the patient from the central tube 11. Because the second linking tube 30 is flexible, the second linking tube 30 can be coordinated with the arteries 100, 100a of the patient to bend appropriately to fasten to the arteries 100, 100a, and the second linking tube 30 with toughness can also support the vascular wall to prevent the arteries 100, 100a from collapsing.

In one embodiment of the present invention, the third linking tube 40 is a tube structure that is flexible. The third linking tube 40 is connected to the second end 16 and connected to the annular tube 12. The third linking tube 40 is used for allowing the waste and urea in solution in the body fluids to flow into the pelvis 210 of the kidney 200 from the annular tube 12 or to flow into the external catheter 500a. The third linking tube 40 includes a third linking tube output 41. The third linking tube output 41 is located at one end of the third linking tube 40, and this end is away from the second end 16. The third linking tube output 41 is used for connecting to the pelvis 210 of the kidney 200 or to the external catheter 500a such that the waste and the urea in solution in the body fluids will flow into the pelvis 210 of the kidney 200 from the annular tube 12 or flow into the external catheter 500a. Because the third linking tube 40 is flexible, the third linking tube 40 can be coordinated with the pelvis 210 of the patient to bend appropriately to fasten to the pelvis 210.

As shown in FIG. 3, FIG. 5 and FIG. 9, in one embodiment of the present invention, the first electrode 51 and the second electrode 52 are used for forming an electric field 50. The electric field 50 covers the central tube 11 to increase the flow rate of the blood and the body fluids in the central tube 11. The first electrode 51 is an annular negative electrode and is located at the first end 15 and around the central tube 11. The second electrode 52 is an annular positive electrode and is located at the second end 16 and around the central tube 11. An electric field 50 is formed between the first electrode 51 and the second electrode 52, and the electric field 50 has a high voltage and low and direct current. The electric field 50 covers the central tube 11 and provides the function of acceleration to the central tube 11; the function of acceleration causes the blood and the body fluids in the central tube 11 to flow faster along an electric field acceleration direction A such that the blood and the body fluids in the central tube 11 will flow faster to the second end 16, such that the urea and the waste in solution in the body fluids can permeate into the annular tube 12 via the dialysis holes 14 more efficiently. Furthermore, the current of the electric field 50 of the present invention is very small, such that the current will not affect the human body.

As shown in FIG. 1, FIG. 3 and FIG. 9, in one embodiment of the present invention, the sensor 70 is located at the position where the second end 16 is connected to the second linking tube 30. The sensor 70 is used for sensing specific information (such as the temperature) of the blood which flows from the main tube 10 and transferring the specific information to the two wireless modules 80, 80a. The two wireless modules 80, 80a are used for electrically connecting to an external network device (such as a wireless access point or a computer) to transfer the information of the blood of the patient (such as the temperature sensed by the sensor 70) to the external network device so that healthcare workers can check the blood information of the patient to know the health status of the patient at any time. The wireless module 80 is located at the first end 15 for emitting a wireless signal toward a side of the patient's body (i.e., emitting the wireless signal toward the right side or the left side of the patient's body); the wireless module 80a is located at the position where the second end 16 is connected to the second linking tube 30 for emitting the wireless signal toward the thigh (i.e., emitting the wireless signal downward toward the left or right lower side of the patient's body). Via the different positions of the wireless modules 80, 80a, the wireless modules 80, 80a can emit the wireless signal along many possible directions. Therefore, the wireless connection between the wireless modules 80, 80a and the external device can be stable; if one of the wireless modules ceases to function, the other wireless module can still transfer the wireless signal. However, the amount and the positions of the wireless modules 80, 80a are not limited to the abovementioned design and can be changed according to design requirements.

In one embodiment of the present invention, the power module 90 is a battery with a wireless charging function. The power module 90 is located at the position where the second end 16 is connected to the second linking tube 30. The power module 90 is used for providing power to the first electrode 51, the second electrode 52, the sensor 70, the wireless modules 80, 80a and the controlling module 60. Via the wireless charging function of the power module 90, if a patient who has the hemodialysis device 1 installed in his or her body (for example, as shown in FIG. 1, the hemodialysis device 1 is installed near the kidney 200) needs to charge the power module 90, the patient only needs to position an external wireless charger close to the patient's waist, and the external wireless charger will be electrically connected to the power module 90 of the hemodialysis device 1 to charge the power module 90; similarly, if the hemodialysis device 1 is installed on the outside of the patient's body (as shown in FIG. 8), then the patient still only needs to position the external wireless charger close to the hemodialysis device 1 to charge the power module 90 of the hemodialysis device 1.

In one embodiment of the present invention, the controlling module 60 is located at the first end 15. The controlling module 60 is connected to the first electrode 51, the second electrode 52, the sensor 70, the wireless modules 80, 80a and the power module 90. The controlling module 60 is used for controlling the first electrode 51, the second electrode 52, the sensor 70, the wireless modules 80, 80a and the power module 90 such that the first electrode 51 can coordinate with the second electrode 52 to generate the electric field 50; the controlling module 60 can also cause the sensor 70 to sense the information (such as the temperature) of the blood which flows from the main tube 10 and transfer the information of the blood to the two wireless modules 80, 80a so that the wireless modules 80, 80a can access the external network device to transfer the information; the controlling module 60 can also analyze the information which is sensed by the sensor 70 to adjust the acceleration performance of the electric field 50 according to the information; the controlling module 60 can also control the power module 90 to provide power or to be charged by the external power device. However, the position of the controlling module 60 is not limited to being at the first end 15; it can also be located at the second end 16.

In one embodiment of the present invention, when a patient with kidney failure needs to use the hemodialysis devices 1, 1a of the present invention to replace the function of the failed kidney to metabolize the urea and the waste in the patient's body, as shown in FIG. 1 to FIG. 2, the patient can ask the doctor to install the two hemodialysis devices 1, 1a into the patient's body to replace the nonfunctioning kidney to metabolize the urea and the waste in the patient's body. However, the method of using the hemodialysis devices 1, 1a is not limited to installation in the body, and the amount of the hemodialysis devices 1, 1a is not limited to two; for example, as shown in FIG. 8, if the patient has used the traditional blood hemodialysis therapy, an external catheter 500 must be installed in the hand of the patient for traditional blood hemodialysis; therefore, the patient can also connect a hemodialysis device 1 to the external catheter 500 on the outside of the body of the patient to replace the nonfunctioning kidney to metabolize the urea and the waste in the body of the patient.

When the doctor operates on the patient to install the hemodialysis devices 1, 1a in the patient's body, the doctor can take a few saphenous veins from the lower limb of the patient, use the vascular walls of the saphenous veins to cover the hemodialysis devices 1, 1a, and install the hemodialysis devices 1, 1a covered by the saphenous veins in the patient's body; because the saphenous veins are generated by the body of the patient, no allergic reaction or rejection will be caused in the patient's body by the hemodialysis devices 1, 1a, which are covered by the saphenous veins. When the two hemodialysis devices 1, 1 a are installed in the human body, as shown in FIG. 1 to FIG. 3, the first fastening tubes 22 of the hemodialysis devices 1, 1a are fastened and connected to the vein 100′ to receive the blood, the body fluids, and the urea and waste in solution in the body fluids from the vein 100′ of the patient; the second fastening tubes 31 of the hemodialysis devices 1, 1 a are fastened and connected to the artery 100 such that the clean blood will flow into the artery 100 of the patient; the third linking tubes 40 of the hemodialysis devices 1, 1a are connected to the pelvis 210 of the kidney 200 such that the body fluids, the waste and the urea will flow into the pelvis 210 of the kidney 200 from the annular tube 12.

As shown in FIG. 2, FIG. 3 and FIG. 5, when the blood and the body fluids, and the urea and waste in solution in the body fluids of the patient, flow in the vein 100′ and contact the hemodialysis device 1, a pushing force will be generated in the main tube 10 along the electric field acceleration direction A because the electric field 50 formed by the first electrode 51 and the second electrode 52 has an acceleration function for the liquid; the pushing force creates a suction effect in the first linking tube 20 to attract the liquid to flow into the main tube 10 such that the blood and the body fluids in the first linking tube 20 will flow faster to the main tube 10. Therefore, the blood and the body fluids in the vein 100′ will be affected by the pushing force and flow into the first fastening tube 22 of the hemodialysis device 1. Because the hemodialysis device 1a and the hemodialysis device 1 are two symmetrical structures with the same units and same function, the hemodialysis device 1a can also generate the electric field 50 to cause the liquid to flow faster such that the blood and the body fluids in the vein 100′ flow into the first fastening tube 22 of the hemodialysis device 1a.

After the blood, the body fluids and the urea and waste in solution in the body fluids of the patient flow into the first fastening tube 22 of the hemodialysis device 1, the blood, the body fluids and the urea and waste in solution in the body fluids will flow to the connecting unit 21. Via the funnel structure of the connecting unit 21, the great amount of blood and body fluid in the funnel of the connecting unit 21 will exert pressure on the blood and the body fluids in the main tube 10 and cause the blood and the body fluids in the main tube 10 to flow smoothly.

As shown in FIG. 3 to FIG. 7, after the blood, the body fluids, and the urea and waste in solution in the body fluids of the patient flow into the central tube 11 from the connecting unit 21, the electric field 50 covered by the central tube 11 will provide an electric field acceleration effect on the central tube 11 and on the blood, the body fluids, and the urea and waste in solution in the body fluids in the central tube 11 such that the blood, the body fluids, and the urea and waste in solution in the body fluids in the central tube 11 will flow more quickly along the electric field acceleration direction A. When the blood, the body fluids, and the urea and waste in solution in the body fluids in the central tube 11 pass the dialysis holes 14, the liquid pressure of the fast flowing liquid caused by the electric field 50 will cause the body fluids and the urea and waste in solution in the body fluids to pass through the dialysis holes 14 and flow into the waste passage 121 along the direction of waste flow C; meanwhile, because the diameter of each dialysis hole 14 is less than the average diameter of the leucons, the average diameter of the red blood cells, and the average diameter of the thrombocytes, the major components of the blood (such as the leucons, the red blood cells and the thrombocytes) cannot pass through the dialysis holes 14 to enter the waste passage 121, and only the body fluids and the urea and waste in solution in the body fluids can pass through the dialysis holes 14 to enter the waste passage 121; in other words, the body fluids and the urea and waste in solution in the body fluids are separated from the blood, and the clean blood flows into the second linking tube 30 along the direction of the blood flow B, while the body fluids and the urea and waste in solution in the body fluids flow into the waste passage 121 and enter the third linking tube 40 along the direction of outflow D.

As shown in FIG. 1 to FIG. 3, FIG. 6, FIG. 7 and FIG. 9, after the clean blood flows into the second linking tube 30 along the direction of the blood flow B, the clean blood will flow back to the artery 100 of the patient from the second fastening tube 31; meanwhile, the sensor 70 located at the position where the second end 16 is connected to the second linking tube 30 will sense the information (such as the temperature) of the clean blood and transfer the information to the wireless modules 80, 80a so that the wireless modules 80, 80a can access and send the information to the external network device so that healthcare workers can check the information of the blood and the body fluids of the patient to check the health and body status of the patient; furthermore, the controlling module 60 can also analyze the information sensed by the sensor 70 to adjust the acceleration performance of the electric field 50 according to that information and thereby to change the flow rate of the liquid in the main tube 10.

Furthermore, after the body fluids and the urea and waste in solution in the body fluids enter the third linking tube 40 along the direction of outflow D and flow into the pelvis 210 of the kidney 200 from the third linking tube output 41, the body fluids and the urea and waste in solution in the body fluids which flow into the pelvis 210 will enter the bladder 400 via the ureter 300 connected to the kidney 200 and the bladder 400, and the patient will excrete the body fluids and the urea and waste in solution in the body fluids from the bladder 400. Therefore, the hemodialysis devices 1, 1a installed in the body of the patient separate the clean blood from the urea and waste in solution in the body fluids, retain the clean blood in the body of the patient, and cause the toxic urea and waste to be excreted to the outside of the body via the original excretory organs of the patient. Furthermore, when the body fluids and the urea and waste in solution in the body fluids flow into the pelvis 210 of the kidney 200 from the third linking tube output 41, the body fluids will continuously exert pressure on and massage the pelvis 210 such that if the pelvis 210 is damaged, the pelvis 210 may gradually restore its function because of the pressing and massaging caused by the body fluid.

If the patient has used traditional blood hemodialysis therapy, an external catheter 500 (which is connected to the artery 100a) will be installed on the hand of the patient for the traditional blood hemodialysis therapy; therefore, as shown in FIG. 8, the patient can connect the first fastening tube 22 and the second fastening tube 31 of the hemodialysis device 1 and connect them to the external catheter 500 to receive the blood, the body fluids and the urea and waste in solution in the body fluids of the patient from the artery 100a, and the patient can connect the third linking tube 40 of the hemodialysis device 1 to the external catheter 500a. As indicated by the abovementioned description, the hemodialysis device 1 can receive the blood, the body fluids, and the urea and waste in solution in the body fluids of the patient from the first fastening tube 22 such that the blood will be separated from the urea and waste in solution in the body fluids of the patient. Then the clean blood will flow into the external catheter 500 via the second fastening tube 31 and flow into the body of the patient via the external catheter 500, and the toxic urea and waste will flow into the external catheter 500a via the third linking tube 40 and flow to an external urine bag. Therefore, the hemodialysis device 1 installed on the outside of the body of the patient can still separate the blood and the urea and waste in solution in the body fluids, retain the clean blood in the body of the patient, and cause the toxic urea and waste to be excreted to the outside of the body.

It is to be known that, as shown in FIG. 3, FIG. 7 and FIG. 9, although the liquid pressure of the fast flowing liquid caused by the electric field 50 of the present invention causes the body fluids and the urea and waste in solution in the body fluids to pass through the dialysis holes 14 and flow into the waste passage 121 along the direction of waste flow C, the force of the flowing blood of the patient is enough to cause the body fluids and the urea and waste in solution in the body fluids to pass through the dialysis holes 14 and flow into the waste passage 121 along the direction of waste flow C; therefore, even if the first electrode 51 and second electrode 52 malfunction such that the electric field 50 is disabled or if the power module 90 has no power, the force of the flowing blood of the patient is sufficient to cause the body fluids and the urea and waste in solution in the body fluids to pass through the dialysis holes 14 and flow into the waste passage 121 along the direction of waste flow C. The difference in whether the electric field 50 functions is that, when the electric field 50 functions normally, the efficiency of the flow of the body fluids and the urea and waste in solution in the body fluids for passing through the dialysis holes 14 is greater, and the flow rate of the blood, the body fluids and the urea and waste in solution in the body fluids is higher, such that the circulatory system and the heart of the patient will not bear any additional burden. If the electric field 50 does not function, the efficiency of the body fluids and the urea and waste in solution in the body fluids for passing through the dialysis holes 14 will be slower, and the flow rate of the blood, the body fluids and the urea and waste in solution in the body fluids in the central tube 11 will be lower, such that the rate of blood circulation of the patient will become slightly lower and the heart of the patient will experience some discomfort.

Via the hemodialysis devices 1, 1a of the present invention, the clean blood can be separated from the toxic urea and waste while the clean blood is retained in the body and the toxic urea and waste are excreted to the outside of the body; therefore, the hemodialysis devices 1, 1a can replace a failed kidney of the patient to metabolize waste and thereby to prevent uremia. Furthermore, the hemodialysis devices 1, 1a of the present invention can be installed in the body of the patient without causing an allergic reaction or organ rejection, such that the daily life of the patient will not be affected by the hemodialysis devices 1, 1a and the patient can still maintain his or her original lifestyle. Furthermore, the hemodialysis devices 1, 1a of the present invention use the force of the flowing blood of the patient to cause the urea and the waste to pass through the dialysis holes 14 to separate the blood from the urea and the waste; therefore, if the hemodialysis devices 1, 1a are connected to the arteries 100, 100a or the vein 100′ of the patient, the hemodialysis devices 1, 1a can separate the blood from the urea and the waste; in other words, if the hemodialysis devices 1, 1a are installed on the artery 100 of the patient or on the vein 100′ in the body of the patient, or installed on the external catheter 500 connected to the artery 100a, the hemodialysis devices 1, 1a can continuously separate the blood from the urea and the waste. Therefore, via the hemodialysis devices 1, 1a of the present invention, a patient with kidney failure does not need to wait for a kidney transplant, nor does the patient need to use the traditional peritoneal dialysis therapy, which would affect the daily lifestyle of the patient and place the patient at risk of viral infection and peritonitis, nor does the patient need to use the tradition blood dialysis therapy, which would require time-consuming visits to the hospital every week, such that the function of saving time can be achieved, and the hemodialysis devices 1, 1a installed in the body of the patient can prevent the problem that the patient must be given injections for the tradition blood dialysis therapy, which may easily cause the artery to be blocked.

In summary, regardless of purposes, means and effectiveness, this invention is quite different from the known technology and should merit the issuing of a new patent. However, it is noted that many of the above-mentioned embodiments are only for illustrative purposes.

What is claimed is:

1. A hemodialysis device, comprising:
a main tube, comprising:
a central tube, comprising at least one blood passage;
an annular tube, comprising a waste passage, wherein the waste passage is around the central tube;
a plurality of dialysis holes, located on the central tube,
wherein the at least one blood passage is connected to the waste passage via the plurality of dialysis holes;
a first end; and
a second end, wherein the first end and the second end are two opposite ends of the main tube;
a first linking tube, connected to the first end and to the central tube;
a second linking tube, connected to the second end and to the central tube; and
a third linking tube, connected to the second end and to the annular tube.

2. The hemodialysis device as claimed in claim 1, further comprising a first electrode and a second electrode, wherein the first electrode is located at the first end and the second electrode is located at the second end; the first electrode and the second electrode form an electric field.

3. The hemodialysis device as claimed in claim 2, further comprising a controlling module, wherein the controlling module is electrically connected to the first electrode and the second electrode.

4. The hemodialysis device as claimed in claim 3, further comprising a sensor, wherein the controlling module is electrically connected to the sensor.

5. The hemodialysis device as claimed in claim 4, further comprising a wireless module, wherein the controlling module is electrically connected to the wireless module.

6. The hemodialysis device as claimed in claim 5, further comprising a power module, wherein the controlling module is electrically connected to the power module.

7. The hemodialysis device as claimed in claim 6, wherein the first linking tube further comprises a connecting unit, and the connecting unit is connected to the main tube.

8. The hemodialysis device as claimed in claim 7, wherein the first linking tube further comprises a first fastening tube, the first fastening tube is located at one end of the first linking tube, and the end of the first linking tube is opposite to the connecting unit.

9. The hemodialysis device as claimed in claim 8, wherein the second linking tube further comprises a second fastening tube; the second fastening tube is located at one end of the second linking tube, and the end of the second linking tube is away from the main tube.

10. The hemodialysis device as claimed in claim 9, wherein the connecting unit is shaped as a funnel.
